(19) United States
(12) Patent Application Publication
(10) Pub. No.: US 2024/0207291 A1
(43) Pub. Date: Jun. 27, 2024

(54) METHODS FOR TREATING POXVIRUS INFECTIONS

(71) Applicant: Gilead Sciences, Inc., Foster City, CA (US)

(72) Inventor: John Philip Bilello, Redwood City, CA (US)

(21) Appl. No.: 18/512,088

(22) Filed: Nov. 17, 2023

Related U.S. Application Data
(60) Provisional application No. 63/426,536, filed on Nov. 18, 2022.

(51) Int. Cl. A61K 31/675; A61P 31/20

(52) U.S. Cl. CPC A61K 31/675; A61P 31/20

(57) ABSTRACT

The present disclosure relates to methods for treating poxvirus infections using a compound of Formula I:

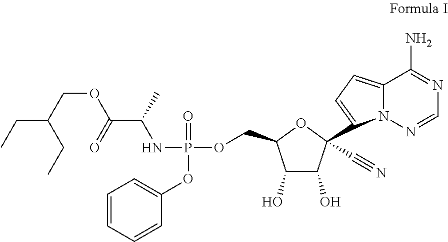 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

or a pharmaceutically acceptable salt or deuterated analog thereof.

### CROSS REFERENCE TO RELATED APPLICATIONS

This application claims priority to U.S. Provisional Application No. 63/426,536, filed Nov. 18, 2022, which is hereby incorporated by reference in its entirety of all purposes.

### FIELD OF THE INVENTION

The present disclosure relates to methods for treating poxvirus infections.

### BACKGROUND

Poxviruses, which are members of the Poxviridae family, exist throughout the world and can infect humans and many other types of animals. Poxvirus infections can arise from contact with contaminated animals, people, or materials, and typically result in the formation of skin lesions. There is a need for compounds and methods for treating poxvirus infections, for example orthopoxvirus, parapoxvirus, molluscipoxvirus, yatapoxvirus, capripoxvirus, suipoxvirus, leporipoxvirus, and avipoxvirus infections.

One such poxvirus, Monkeypox virus, was first discovered in laboratory monkeys in 1958 and can infect both animals and humans. Monkeypox virus belongs to the orthopoxvirus genus. The orthopoxvirus genus also includes variola virus (which causes smallpox), vaccinia virus (used in the smallpox vaccine), and cowpox virus. Since the first human case of monkeypox infection was recorded in 1970, the majority of reported cases have been in Democratic Republic of the Congo and other central and western African countries. Recently, multiple cases have been reported in countries that do not normally report monkeypox infections, including Australia and countries in Europe and North America.

### SUMMARY

Provided are methods for the treatment or prevention of infections caused by a poxvirus.

Provided is a method of treating or preventing a poxvirus infection in a patient in need thereof, wherein the method includes administering to the patient a therapeutically effective amount of a compound of Formula I:

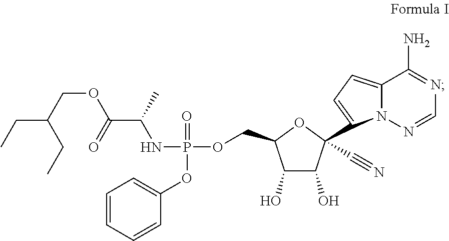 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

or a pharmaceutically acceptable salt or deuterated analog thereof.

Also provided is a method of treating or preventing a poxvirus infection in a patient in need thereof, wherein the method includes administering to the patient a pharmaceutical composition including a compound of Formula I, or a pharmaceutically acceptable salt or deuterated analog thereof, and one or more pharmaceutically acceptable carriers.

Also provided is a compound of Formula I, or a pharmaceutically acceptable salt or deuterated analog thereof, for use in treating or preventing a poxvirus infection.

Also provided is a use of a compound of Formula I, or a pharmaceutically acceptable salt or deuterated analog thereof, in the preparation of a medicament for treating or preventing a poxvirus infection.

Also provided is a kit that includes a compound of Formula I, or a pharmaceutically acceptable salt or deuterated analog thereof, and directions for their use in treating or preventing a poxvirus infection.

### DETAILED DESCRIPTION

Unless stated otherwise, the following terms and phrases as used herein are intended to have the following meanings:

When trade names are used herein, applicants intend to independently include the trade name product and the active pharmaceutical ingredient(s) of the trade name product.

The term “patient,” as used herein, refers to any animal including mammals such as mice, rats, other rodents, rabbits, dogs, cats, swine, cattle, sheep, horses, primates, and humans.

The term “treating”, as used herein, unless otherwise indicated, means reversing, alleviating, or inhibiting the progress of the disorder or condition to which such term applies, or one or more symptoms of such disorder or condition. The term “treatment”, as used herein, refers to the act of treating, as “treating” is defined immediately above.

“Prevention” or “preventing” means any treatment of a disease or condition that causes the clinical symptoms of the disease or condition not to develop. The compounds and compositions disclosed herein may, in some embodiments, be administered to a subject (including a human) who is at risk of having the disease or condition. As used herein, the terms “preventing” and “prevention” encompass the administration of a compound, composition, or pharmaceutically acceptable salt according to the embodiments disclosed herein pre- or post-exposure of the individual to a virus, but before the appearance of symptoms of the viral infection, and/or prior to the detection of the virus in the blood. The terms also refer to prevention of the appearance of symptoms of the disease and/or to prevent the virus from reaching detectible levels in the blood. The terms include both pre-exposure prophylaxis (PrEP), as well as post-exposure prophylaxis (PEP) and event-driven or “on demand” prophylaxis. The terms also refer to prevention of perinatal transmission of a virus from mother to baby, by administration to the mother before giving birth and to the child within the first days of life. The terms also refer to prevention of transmission of a virus through blood transfusion.

The term “therapeutically effective amount”, as used herein, is the amount of an active ingredient that is needed to provide a desired level of drug in the bloodstream of a subject to be treated to give an anticipated physiological response or desired biological effect when such a compound is administered by the chosen route of administration. The precise amount will depend upon numerous factors, for example the particular compound, the specific activity of the compound, the delivery device employed, the physical characteristics of the compound, its intended use, as well as patient considerations such as severity of the disease state, patient cooperation, etc., and can readily be determined by one skilled in the art based upon the information provided herein.

Reference will now be made in detail to certain embodiments of the invention, examples of which are illustrated in the accompanying description. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention.

Provided is a method of treating a poxvirus infection in a patient in need thereof, wherein the method comprises administering to the patient a therapeutically effective amount of a compound of Formula I:

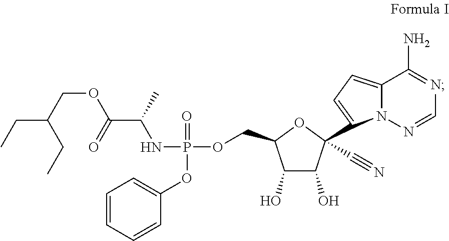 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

or a pharmaceutically acceptable salt or deuterated analog thereof.

Also provided is a method of preventing a poxvirus infection in a patient in need thereof, wherein the method comprises administering to the patient a therapeutically effective amount of a compound of Formula I, or a pharmaceutically acceptable salt or deuterated analog thereof.

In some embodiments, the compound of Formula I is a compound of Formula Ia:

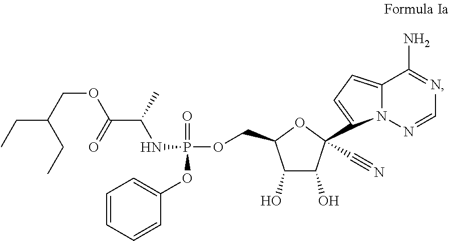 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

or a pharmaceutically acceptable salt or deuterated analog thereof. In some embodiments, the compound of Formula I is the compound of Formula Ia.

The compound of Formula Ia is also known as remdesivir and GS-5734. The IUPAC name of the compound of Formula Ia is (S)-2-ethylbutyl 2-(((S)-(((2R,3S,4R,5R)-5-(4-aminopyrrolo[2,1-f][1,2,4]triazin-7-yl)-5-cyano-3,4-dihydroxytetrahydrofuran-2-yl)methoxy)(phenoxy)phosphoryl)amino)propanoate with CAS Registry No. 1809249-37-3. The compound of Formula Ia is disclosed in U.S. Pat. Nos. 9,724,360; 10,065,958; and 10,695,361; the contents of each are incorporated herein in their entireties.

In some embodiments, the compound of Formula I is a compound of Formula Ib:

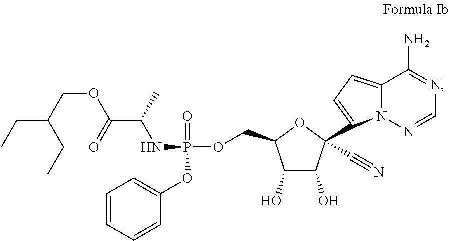 [SMILES: CCC(CC)COC(=O)[C@H](C)N[P@@](=O)(OC[C@H]1O[C@@](C#N)(c2ccc3c(N)ncnn23)[C@H](O)[C@@H]1O)Oc1ccccc1]

or a pharmaceutically acceptable salt or deuterated analog thereof. In some embodiments, the compound of Formula I is the compound of Formula Ib.

In some embodiments, a compound of Formula I is administered. In some embodiments, a compound of Formula Ia is administered. In some embodiments, a compound of Formula Ib is administered.

The methods described herein can be used to treat or prevent a poxvirus infection. In some embodiments, the poxvirus infection is an orthopox virus infection. In some embodiments, the poxvirus infection is a camelpox virus infection, cowpox virus infection, ectromelia virus infection, horsepox virus infection, monkeypox virus infection, raccoonpox virus infection, skunkpox virus infection, taterapox virus infection, uasin gishu virus infection, vaccinia virus infection, variola virus infection, or volepox virus infection.

In some embodiments, the poxvirus infection is a vaccinia virus infection.

In some embodiments, the poxvirus infection is a monkeypox virus infection. The methods described herein can be used to treat or prevent an infection caused by any strain of monkeypox virus. In some embodiments, the poxvirus infection is caused by a West African strain of monkeypox virus. In some embodiments, the poxvirus infection is caused by a Congo Basin strain of monkeypox virus.

In some embodiments, the poxvirus infection is a parapoxvirus infection. In some embodiments, the poxvirus infection is bovine papular stomatitis virus infection, orf virus infection, pseudocowpox virus infection, parapoxvirus of red deer infection, or squirrel parapoxvirus infection. In some embodiments, the poxvirus infection is camel contagious ecthyma (Ausdyk) virus infection, chamois contagious ecthyma virus infection, parapoxvirus of reindeer virus infection, or sealpox virus infection.

In some embodiments, the poxvirus infection is a molluscipoxvirus infection. In some embodiments, the poxvirus infection is a molluscum contagiosum infection.

In some embodiments, the poxvirus infection is a yatapoxvirus infection. In some embodiments, the poxvirus infection is a Tanapox, Yaba-like disease virus infection or yaba monkey tumor virus infection.

In some embodiments, the poxvirus infection is a capripoxvirus infection. In some embodiments, the poxvirus infection is a sheeppox virus infection, goatpox virus infection, or lumpy skin disease virus infection.

In some embodiments, the poxvirus infection is a suipoxvirus infection. In some embodiments, the poxvirus infection is a swinepox virus infection.

In some embodiments, the poxvirus infection is a leporipoxvirus infection. In some embodiments, the poxvirus infection is a myxoma virus infection, shope fibroma virus (rabbit fibroma) infection, squirrel fibroma virus infection, or hare fibroma virus infection.

In some embodiments, the poxvirus infection is an avipoxvirus infection. In some embodiments, the poxvirus infection is canarypox virus infection, fowlpox virus infection, juncopox virus infection, mynahpox virus infection, pigeonpox virus infection, psittacinepox virus infection, quailpox virus infection, sparrowpox virus infection, starlingpox virus infection, or turkeypox virus infection. In some embodiments, the poxvirus infection is crowpox virus infection, peacockpox virus infection, or penguinpox virus infection.

Also provided is a method of treating a poxvirus infection in a patient in need thereof, wherein the method comprises administering to the patient a pharmaceutical composition comprising a compound of Formula I, or a pharmaceutically acceptable salt or deuterated analog thereof, and one or more pharmaceutically acceptable carriers.

Also provided is a method of preventing a poxvirus infection in a patient in need thereof, wherein the method comprises administering to the patient a pharmaceutical composition comprising a compound of Formula I, or a pharmaceutically acceptable salt or deuterated analog thereof, and one or more pharmaceutically acceptable carriers.

In some embodiments, the pharmaceutical composition comprises a compound of Formula Ia, or a pharmaceutically acceptable salt or deuterated analog thereof. In some embodiments, the pharmaceutical composition includes a compound of Formula Ib, or a pharmaceutically acceptable salt or deuterated analog thereof.

Pharmaceutically acceptable carriers can be selected in accord with ordinary practice. For example, tablets can contain excipients, glidants, fillers, binders and the like. Aqueous compositions can be prepared in sterile form, and when intended for delivery by other than oral administration generally can be isotonic. Pharmaceutical compositions described herein can optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the pharmaceutical composition can range from 3 to 11, but is ordinarily 7 to 10. In some embodiments, the pH of the pharmaceutical composition ranges from 2 to 5, but is ordinarily 3 to 4.

The pharmaceutical compositions described herein include at least one active ingredient (e.g. a compound of Formula I, Formula Ia, Formula Ib, or pharmaceutically acceptable salt or deuterated analog thereof) together with one or more pharmaceutically acceptable carriers and optionally other therapeutic ingredients, such as one or more additional therapeutic ingredients described herein. The one or more carriers are pharmaceutically acceptable, that is, the one or more carriers are compatible with the other ingredients of the pharmaceutical composition and physiologically innocuous to the recipient thereof.

The pharmaceutical compositions may be in unit dosage form and may be prepared by any method well-known in the pharmaceutical arts. General techniques and formulations can be found in Remington's Pharmaceutical Sciences (Mack Publishing Co., Easton, PA). Such methods include, for example, bringing into association the active ingredient with one or more pharmaceutically acceptable carriers. The pharmaceutical compositions described herein can be prepared, for example, by uniformly and intimately bringing into association the active ingredient with liquid carriers or finely divided solid carriers or both, and then, if necessary, shaping the product.

Pharmaceutical compositions suitable for oral administration may be in the form of discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

For oral use, the pharmaceutical compositions described herein can be in the form of, for example, tablets, troches, lozenges, aqueous or oil suspensions, dispersible powders or granules, emulsions, hard or soft capsules, syrups or elixirs. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation.

For example, a pharmaceutical composition in the form of a tablet can contain the active ingredient in admixture with one or more pharmaceutically acceptable carriers suitable for manufacture of tablets are acceptable. These carriers may include, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc.

A tablet can be made by compression or molding with one or more pharmaceutically acceptable carriers. Compressed tablets may be prepared by compressing in a suitable machine the active ingredient in a free-flowing form such as a powder or granules, e.g., mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally can be formulated so as to provide slow or controlled release of the active ingredient therefrom. For example, tablets may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Pharmaceutical compositions described herein may also be in the form of hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of a pharmaceutical composition described herein can contain the active ingredient in admixture with one or more pharmaceutically acceptable carriers suitable for the manufacture of aqueous suspensions. Such carriers include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin and Captisol (=Sulfobutyl ether beta-cyclodextrin; SEB-beta-CD).

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Pharmaceutical compositions described herein, in the form of dispersible powders and granules suitable for preparation of an aqueous suspension by the addition of water, can include the active ingredient in admixture with a dispersing or wetting agent, a suspending agent, and one or more preservatives. Suitable dispersing or wetting agents and suspending agents are exemplified by those described above. Additional excipients, for example sweetening, flavoring and coloring agents, may also be present.

The pharmaceutical compositions described herein may also be in the form of an oil-in-water emulsion. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions described herein may be in the form of a sterile injectable preparation, such as a sterile injectable aqueous or oleaginous suspension. This suspension may be formulated according to the known procedures using, for example, suitable dispersing or wetting agents and suspending agents described above. The sterile injectable preparation may also be a sterile solution or suspension in a non-toxic, parenterally acceptable diluent or solvent, such as a solution in 1,3-butane-diol, or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of active ingredient that may be combined with the carrier material to form a single dosage form can vary depending upon the patient and the particular mode of administration. For example, a time-release formulation intended for oral administration to humans may contain approximately 1 to 1000 mg of active material compounded with an appropriate and convenient amount of carrier material, which may vary from 5 to 95% of the total compositions (weight:weight). The pharmaceutical composition can be prepared to provide easily measurable amounts for administration. For example, an aqueous solution intended for intravenous infusion may contain from 3 to 500 μg of the active ingredient per milliliter of solution, so that infusion of a suitable volume at a rate of 30 mL/hr can occur.

Pharmaceutical compositions suitable for topical administration in the mouth include lozenges containing the active ingredient in a flavored basis, usually sucrose and acacia or tragacanth; pastilles containing the active ingredient in an inert basis such as gelatin and glycerin, or sucrose and acacia; and mouthwashes containing the active ingredient in a suitable liquid carrier.

The pharmaceutical compositions can be in a unit-dose or multi-dose form, for example sealed ampoules and vials, and may be stored in a freeze-dried (lyophilized) condition requiring only the addition of the sterile liquid carrier, for example water for injection, immediately prior to use. Extemporaneous injection solutions and suspensions can be prepared from sterile powders, granules and tablets of the kind previously described. Preferred unit dosage formulations are those containing a daily dose or unit daily sub-dose, as described herein, or an appropriate fraction thereof, of the active ingredient.

It should be understood that in addition to the ingredients particularly mentioned above, the pharmaceutical compositions described herein may include other agents conventional in the art having regard to the type of pharmaceutical composition in question, for example, those suitable for oral administration may include flavoring agents.

Also described herein are veterinary compositions that include at least one active ingredient as described herein, together with a veterinary carrier therefor. Veterinary carriers are materials useful for the purpose of administering the composition and may be solid, liquid or gaseous materials which are otherwise inert or acceptable in the veterinary arts and are compatible with the active ingredient. These veterinary compositions may be administered orally, parenterally, or by any other desired route.

The compounds and pharmaceutical compositions described herein can be administered by any route appropriate to the condition to be treated or prevented. Suitable routes include oral, rectal, nasal, pulmonary, topical (including buccal and sublingual), vaginal and parenteral (including subcutaneous, intramuscular, intravenous, intradermal, intrathecal and epidural), and the like. It will be appreciated that the preferred route may vary with, for example, the condition of the recipient. An advantage of the compounds described herein is that they are orally bioavailable and can be dosed orally.

In some embodiments, a compound described herein (for example, a compound of Formula I, Formula Ia, or Formula Ib) or the pharmaceutically acceptable salt or deuterated analog thereof is administered orally. In some embodiments, a compound described herein or the pharmaceutically acceptable salt or deuterated analog thereof is administered parenterally.

In some embodiments, a pharmaceutical composition described herein is administered orally. In some embodiments, a pharmaceutical composition described herein is administered parenterally.

In some embodiments, the patient is a human. In the methods described herein, the compounds and pharmaceutical compositions described herein can be administered at any time to a human who may come into contact with humans suffering from a poxvirus infection or who is already suffering from a poxvirus infection. In some embodiments, the compounds described herein can be administered prophylactically to humans coming into contact with humans suffering from a poxvirus infection or at risk of coming into contact with humans suffering from a poxvirus infection, e.g. healthcare providers. In some embodiments, administration of the compounds described herein can be to humans testing positive for an infection but not yet showing symptoms of a poxvirus infection. In some embodiments, administration of the compounds described herein can be to humans upon commencement of symptoms of a poxvirus infection.

In some embodiments, the methods described herein include event-driven administration of a compound of a pharmaceutical composition described herein to the subject. As used herein, the terms “event-driven” or “event-driven administration” refer to administration of the compound or pharmaceutical composition (1) prior to an event (e.g., 2 hours, 1 day, 2 days, 5 day, or 7 or more days prior to the event) that would expose the individual to a poxvirus (or that would otherwise increase the individual's risk of acquiring a poxvirus infection); and/or (2) during an event (or more than one recurring event) that would expose the individual to a poxvirus (or that would otherwise increase the individual's risk of acquiring a poxvirus infection); and/or (3) after an event (or after the final event in a series of recurring events) that would expose the individual to a poxvirus (or that would otherwise increase the individual's risk of acquiring a poxvirus infection). In some embodiments, the event-driven administration is performed pre-exposure of the subject to a poxvirus. In some embodiments, the event-driven administration is performed post-exposure of the subject to a poxvirus. In some embodiments, the event-driven administration is performed pre-exposure of the subject to a poxvirus and post-exposure of the subject to a poxvirus.

In some embodiments, the compound or pharmaceutical composition is administered before exposure of the subject to a poxvirus. In some embodiments, the compound or pharmaceutical composition is administered before and after exposure of the subject to a poxvirus. In some embodiments, the compound or pharmaceutical composition is administered after exposure of the subject to a poxvirus.

An example of event-driven dosing regimen includes administration of a compound or pharmaceutical composition described herein within 24 to 2 hours prior to exposure to a poxvirus, followed by administration of the compound or pharmaceutical composition every 24 hours during the period of exposure, followed by a further administration of the compound or pharmaceutical composition after the last exposure, and one last administration of the compound or pharmaceutical composition 24 hours later.

A further example of an event-driven dosing regimen includes administration of a compound or pharmaceutical composition described herein within 24 hours before exposure to a poxvirus, then daily administration during the period of exposure, followed by a last administration approximately 24 hours later after the last exposure (which may be an increased dose, such as a double dose).

The effective dose of a compound described herein depends at least on the nature of the condition being treated or prevented, toxicity, whether the compound is being used prophylactically or against an active viral infection, the method of delivery, and the pharmaceutical formulation, and will be determined by the clinician using conventional dose escalation studies. It can be expected to be from 0.0001 to 100 mg/kg body weight per day; typically, from 0.01 to 10 mg/kg body weight per day; more typically, from 0.01 to 5 mg/kg body weight per day; most typically, from 0.05 to 0.5 mg/kg body weight per day. For example, the daily candidate dose for an adult human of approximately 70 kg body weight will range from 1 mg to 1000 mg, e.g., between 5 mg and 500 mg, and may take the form of single or multiple doses.

The effective dose of a compound described herein for treating or preventing a poxvirus infection can depend on whether the dose is to be used prophylactically or to treat a human already suffering from a poxvirus infection. Moreover, the dose can depend on whether the human suffering from a poxvirus infection does not yet show symptoms or is already showing symptoms of a poxvirus infection. Larger doses may be necessary for treating humans testing positive for a poxvirus infection and for humans showing symptoms of a poxvirus infection as compared to humans receiving prophylactic administration.

Any suitable period of time for administration of the compounds and pharmaceutical compositions described herein is contemplated. For example, administration can be for from 1 day to 100 days, including 2, 3, 4, 5, 6, 7, 8, 9, 10, 15, 20, 25, 30, 40, 50, 60, 70, 80, or 90 days. The administration can also be for from 1 week to 15 weeks, including 2, 3, 4, 5, 6, 7, 8, 9, 10, 11, 12, 13, or 14 weeks. Longer periods of administration are also contemplated. The time for administration can depend on whether the compound is being administered prophylactically or to treat a human suffering from a poxvirus infection. For example, a prophylactic administration can be for a period of time while the human is in regular contact with other humans suffering from a poxvirus infection, and for a suitable period of time following the last contact with a human suffering from a poxvirus infection. For humans already suffering from a poxvirus infection, the period of administration can be for any length of time necessary to treat the patient and a suitable period of time following a negative test for a poxvirus infection to ensure the poxvirus infection does not return.

In some embodiments, a compound described herein (for example, a compound of Formula I, Formula Ia, or Formula Ib) or the pharmaceutically acceptable salt or deuterated analog thereof is administered once daily. In some embodiments, a compound described herein or the pharmaceutically acceptable salt or deuterated analog thereof is administered once every alternate day. In some embodiments, a compound described herein or the pharmaceutically acceptable salt or deuterated analog thereof is administered once every third day. In some embodiments, a compound described herein or the pharmaceutically acceptable salt or deuterated analog thereof is administered once a week. In some embodiments, a compound described herein or the pharmaceutically acceptable salt or deuterated analog thereof is administered twice a week.

In some embodiments, a compound described herein or the pharmaceutically acceptable salt or deuterated analog thereof is administered once daily for a period of five days. In some embodiments, a compound described herein or the pharmaceutically acceptable salt or deuterated analog thereof is administered once daily for a period of 10 days.

In some embodiments, a pharmaceutical composition described herein is administered once daily. In some embodiments, a pharmaceutical composition described herein is administered once every alternate day. In some embodiments, a pharmaceutical composition described herein is administered once every third day. In some embodiments, a pharmaceutical composition described herein is administered once a week. In some embodiments, a pharmaceutical composition described herein is administered twice a week.

In some embodiments, a pharmaceutical composition described herein is administered once daily for a period of five days. In some embodiments, a pharmaceutical composition described herein is administered once daily for a period of 10 days.

In some embodiments, a compound described herein or the pharmaceutically acceptable salt or deuterated analog thereof is administered at a dosage of 5 mg to 500 mg. In some embodiments, a compound described herein or the pharmaceutically acceptable salt or deuterated analog thereof is administered at a dosage of 5 mg to 300 mg. In some embodiments, 5-300 mg of a compound described herein is administered once daily, e.g., for 6 to 12 days. In some embodiments, 5-300 mg of a compound described herein is administered once daily, e.g., for 5 days or for 10 days. In some embodiments, a compound described herein or the pharmaceutically acceptable salt or deuterated analog thereof is administered at a dosage of 100 mg or 200 mg. In some embodiments, a compound described herein or the pharmaceutically acceptable salt or deuterated analog thereof is administered at a dosage of 100 mg. In some embodiments, a compound described herein or the pharmaceutically acceptable salt or deuterated analog thereof is administered at a dosage of 200 mg. In some embodiments, a compound described herein or the pharmaceutically acceptable salt or deuterated analog thereof is administered at a dosage of 0.1 mg/kg to 15 mg/kg, for example, 0.1 mg/kg to 10 mg/kg. In some embodiments, a compound described herein or the pharmaceutically acceptable salt or deuterated analog thereof is administered at a dosage of 0.1 mg/kg to 15 mg/kg.

The compounds and pharmaceutical compositions described herein can also be used in combination with one or more additional therapeutic agents. Accordingly, also provided herein is a method of treating a poxvirus infection in a patient in need thereof, wherein the method includes administering to the patient a compound described herein or the pharmaceutically acceptable salt or deuterated analog thereof, further comprising administering to the patient a therapeutically effective amount of an additional therapeutic agent. Also provided herein is a method of treating a poxvirus infection in a patient in need thereof, wherein the method comprises administering to the patient a pharmaceutical composition, wherein the pharmaceutical composition includes a compound described herein or pharmaceutically acceptable salt or deuterated analog thereof, and further comprises an additional therapeutic agent.

In some embodiments, the additional therapeutic agent is an antiviral agent. Any suitable antiviral agent can be used in the methods described herein. In some embodiments, the additional therapeutic agent includes 5-substituted 2′-deoxyuridine analogues, nucleoside analogues, pyrophosphate analogues, polymerase inhibitors, nucleoside reverse transcriptase inhibitors, non-nucleoside reverse transcriptase inhibitors, protease inhibitors, integrase inhibitors, entry inhibitors, acyclic guanosine analogues, acyclic nucleoside phosphonate analogues, HCV NS5A inhibitors, NS5B inhibitors, influenza virus inhibitors, interferons, immunostimulators, oligonucleotides, antimitotic inhibitors, or any combination thereof.

In some embodiments, the additional therapeutic agent includes one or more 5-substituted 2′-deoxyuridine analogues. In some embodiments, the one or more additional therapeutic agents include idoxuridine, trifluridine, brivudine [BVDU], or any combination thereof.

In some embodiments, the additional therapeutic agent includes one or more nucleoside analogues. In some embodiments, the additional therapeutic agent includes vidarabine, entecavir (ETV), telbivudine, lamivudine, famciclovir, clevudine, or any combination thereof. In some embodiments, the additional therapeutic agent is favipiravir, ribavirin, galidesivir, or a combination thereof. In some embodiments, the additional therapeutic agent is ß-D-N4-hydroxycytidine.

In some embodiments, the additional therapeutic agent includes one or more pyrophosphate analogues. In some embodiments, the additional therapeutic agent includes foscarnet or phosphonoacetic acid. In some embodiments, the additional therapeutic agents includes foscarnet.

In some embodiments, the additional therapeutic agent includes one or more polymerase inhibitors. In some embodiments, the additional therapeutic agent includes one or more DNA polymerase inhibitors. In some embodiments, the additional therapeutic agent includes cidofovir. In some embodiments, the additional therapeutic agent includes lamivudine.

In some embodiments, the additional therapeutic agent includes one or more nucleoside reverse transcriptase inhibitors. In some embodiments, the additional therapeutic agent includes zidovudine, didanosine, zalcitabine, stavudine, lamivudine, abacavir, emtricitabine, azvudine, or any combination thereof. In some embodiments, the additional therapeutic agent includes sangivamycin, ß-d-N4-Hydroxycytidine (NHC), EIDD-2801, EIDD-1931, or any combination thereof. In some embodiments, the additional therapeutic agent includes MK-4482 (EIDD-2801).

In some embodiments, the additional therapeutic agent includes one or more non-nucleoside reverse transcriptase inhibitors. In some embodiments, the additional therapeutic agent includes nevirapine, delavirdine, efavirenz, etravirine, rilpivirine, doravirine, or any combination thereof.

In some embodiments, the additional therapeutic agent includes one or more nucleoside reverse transcriptase translocation inhibitors. In some embodiments, the additional therapeutic agent is islatravir.

In some embodiments, the additional therapeutic agent includes one or more protease inhibitors. In some embodiments, the additional therapeutic agent includes a HIV protease inhibitor. In some embodiments, the additional therapeutic agent includes saquinavir, ritonavir, indinavir, nelfinavir, amprenavir, lopinavir, atazanavir, fosamprenavir, darunavir, tipranavir, cobicistat, or any combination thereof. In some embodiments, the additional therapeutic agent includes saquinavir, ritonavir, indinavir, nelfinavir, amprenavir, lopinavir, atazanavir, fosamprenavir, darunavir, tipranavir, or any combination thereof. In some embodiments, the additional therapeutic agent includes a HCV NS3/4A protease inhibitor. In some embodiments, the additional therapeutic agent includes voxilaprevir, asunaprevir, boceprevir, paritaprevir, simeprevir, telaprevir, vaniprevir, grazoprevir, ribavirin, danoprevir, faldaprevir, vedroprevir, sovaprevir, deldeprevir, narlaprevir, or any combination thereof. In some embodiments, the additional therapeutic agent includes voxilaprevir, asunaprevir, boceprevir, paritaprevir, simeprevir, telaprevir, vaniprevir, grazoprevir, or any combination thereof.

In some embodiments, the additional therapeutic agent includes one or more integrase inhibitors. In some embodiments, the additional therapeutic agent includes raltegravir, dolutegravir, elvitegravir, abacavir, lamivudine, or any combination thereof. In some embodiments, the additional therapeutic agent includes bictegravir, raltegravir, dolutegravir, cabotegravir, elvitegravir, or any combination thereof. In some embodiments, the additional therapeutic agent includes bictegravir, dolutegravir, and cabotegravir, or any combination thereof. In some embodiments, the additional therapeutic agent includes bictegravir.

In some embodiments, the additional therapeutic agent includes one or more entry inhibitors. In some embodiments, the additional therapeutic agent includes docosanol, enfuvirtide, maraviroc, ibalizumab, fostemsavir, leronlimab, ibalizumab, fostemsavir, leronlimab, palivizumab, respiratory syncytial virus immune globulin, intravenous [RSV-IGIV], varicella-zoster immunoglobulin [VariZIG], varicella-zoster immune globulin [VZIG]), or any combination thereof.

In some embodiments, the additional therapeutic agent includes one or more acyclic guanosine analogues. In some embodiments, the additional therapeutic agent includes acyclovir, ganciclovir, valacyclovir (also known as valaciclovir), valganciclovir, penciclovir, famciclovir, or any combination thereof.

In some embodiments, the additional therapeutic agent includes one or more acyclic nucleoside phosphonate analogues. In some embodiments, the additional therapeutic agent includes cidofovir, emtricitabine, efavirenz, rilpivirine, elvitegravir, or any combination thereof. In some embodiments, the additional therapeutic agent includes cidofovir.

In some embodiments, the additional therapeutic agent includes one or more HCV NS5A or NS5B inhibitors. In some embodiments, additional therapeutic agent includes one or more NS3/4A protease inhibitors. In some embodiments, the additional therapeutic agent includes one or more NS5A protein inhibitors. In some embodiments, the additional therapeutic agent includes one or more NS5B polymerase inhibitors of the nucleoside/nucleotide type. In some embodiments, the additional therapeutic agent includes one or more NS5B polymerase inhibitors of the non-nucleoside type. In some embodiments, the additional therapeutic agent includes daclatasvir, ledipasvir, velpatasvir, ombitasvir, elbasvir, sofosbuvir, bemnifosbuvir, dasabuvir, ribavirin, asunaprevir, simeprevir, paritaprevir, ritonavir, elbasvir, grazoprevir, or any combination thereof. In some embodiments, the additional therapeutic agent includes daclatasvir, ledipasvir, velpatasvir, ombitasvir, elbasvir, sofosbuvir, bemnifosbuvir, dasabuvir, or any combination thereof.

In some embodiments, the additional therapeutic agent is molnupiravir (EIDD-2801), ASC-10, or any combination thereof.

In some embodiments, the additional therapeutic agent includes one or more influenza virus inhibitors. In some embodiments, the additional therapeutic agent includes one or more matrix 2 inhibitors. In some embodiments, the additional therapeutic agent includes amantadine, rimantadine, or any combination thereof. In some embodiments, the additional therapeutic agent includes one or more neuraminidase inhibitors. In some embodiments, the additional therapeutic agent includes include zanamivir, oseltamivir, peramivir, laninamivir octanoate, or any combination thereof. In some embodiments, the additional therapeutic agent includes one or more polymerase inhibitors. In some embodiments, the additional therapeutic agent includes ribavirin, favipiravir, or any combination thereof. In some embodiments, the additional therapeutic agent includes amantadine, rimantadine, arbidol (umifenovir), baloxavir marboxil, oseltamivir, peramivir, ingavirin, laninamivir octanoate, zanamivir, favipiravir, ribavirin, or any combination thereof. In some embodiments, the additional therapeutic agent includes amantadine, rimantadine, zanamivir, oseltamivir, peramivir, laninamivir octanoate, ribavirin, favipiravir, or any combination thereof. In some embodiments, the additional therapeutic agent includes DAS-181 or XC-221.

In some embodiments, the additional therapeutic agent includes one or more interferons. In some embodiments, the additional therapeutic agent includes interferon alfacon 1, interferon alfa 1b, interferon alfa 2a, interferon alfa 2b, pegylated interferon alfacon 1, pegylated interferon alfa 1b, pegylated interferon alfa 2a (PegIFNα-2a), PegIFNα-2b, or any combination thereof. In some embodiments, the additional therapeutic agent includes interferon alfacon 1, interferon alfa 1b, interferon alfa 2a, interferon alfa 2b, pegylated interferon alfa 2a (PegIFNα-2a), PegIFNα-2b, or any combination thereof. In some embodiments, the additional therapeutic agent includes interferon alfacon 1, pegylated interferon alfa 2a (PegIFNα-2a), PegIFNα-2b, ribavirin, or any combination thereof. In some embodiments, the additional therapeutic agent includes pegylated interferon alfa-2a, pegylated interferon alfa-2b, or any combination thereof. In some examples, the additional therapeutic agent includes interferon-beta. In some embodiments, the additional therapeutic agent includes interferon-beta-1a, such as SNG-001. In some embodiments, the additional therapeutic agent includes one or more interferon-inducing agents, such as tilorone hydrochloride. In some embodiments, the additional therapeutic agent includes an IL-17 antagonist, such as ixekizumab. In some embodiments, the additional therapeutic agent includes interferon alfa 2 ligand, secukinumab, IMU-838, vidofludimus, or any combination thereof.

In some embodiments, the additional therapeutic agent includes one or more immunostimulatory agents. In some embodiments, the additional therapeutic agent includes one or more oligonucleotides. In some embodiments, the additional therapeutic agent includes one or more antimitotic inhibitors. In some embodiments, the additional therapeutic agent includes fomivirsen, podofilox, imiquimod, sinecatechins, or any combination thereof. In some embodiments, the additional therapeutic agent includes azoximer bromide or IMM-101.

In some embodiments, the additional therapeutic agent includes one or more anti-inflammatory agents. Any suitable anti-inflammatory agent can be used in the methods described herein. For example, some embodiments, the additional therapeutic agent includes an inhibitor of Bruton tyrosine kinase (BTK, AGMX1, AT, ATK, BPK, IGHD3, IMD1, PSCTK1, XLA; NCBI Gene ID: 695). For example, some embodiments, the additional therapeutic agent includes (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, AZD6738, calquence, danvatirsen, or any combination thereof. In some embodiments, the additional therapeutic agent includes tirabrutinib, ibrutinib, acalabrutinib, or any combination thereof. In some embodiments, the additional therapeutic agent includes tirabrutinib, ibrutinib, or any combination thereof. In some embodiments, the additional therapeutic agent includes one or more receptor tyrosine kinase inhibitors (RTKI). In some embodiments, the additional therapeutic agent includes tyrphostin A9 (A9). In some embodiments, the additional therapeutic agent includes one or more TEK receptor tyrosine kinase inhibitors. In some embodiments, the additional therapeutic agent includes abivertinib maleate (STI-5656). In some embodiments, the additional therapeutic agent includes one or more tyrosine kinase inhibitors, such as masitinib.

In some embodiments, the additional therapeutic agent includes one or more sphingosine kinase-2 (sk2) inhibitors, such as opaganib. In some embodiments the additional therapeutic agent includes one or more kinase inhibitors, such as pacritinib. In some embodiments, the additional therapeutic agent includes one or more Axl tyrosine kinase receptor inhibitors, such as bemcentinib. In some embodiments, the additional therapeutic agent includes one or more FYVE finger phosphoinositide kinase inhibitors. In some embodiments, the additional therapeutic agent includes one or more checkpoint kinase inhibitors, such as prexasertib. In some embodiments, the additional therapeutic agent includes one or more MAP kinase inhibitors, such as KTH-222, ATI-450. In some embodiments, the additional therapeutic agent includes one or more mTOR inhibitors, such as sirolimus. In some embodiments, the additional therapeutic agent includes one or more pi3k/mTOR inhibitors, such as dactolisib. In some embodiments, the additional therapeutic agent includes one or more Hsp90 inhibitors, such as ganetespib, ADX-1612. In some embodiments, the additional therapeutic agent includes one or more MEK inhibitors, such as ATR-002. In some embodiments, the additional therapeutic agent includes one or more topoisomerase II inhibitors, such as etoposide. In some embodiments, the additional therapeutic agent includes one or more exportin 1 inhibitors, such as selinexor, verdinexor. In some embodiments, the additional therapeutic agent includes one or more dual inhibitors of PARP1/2 and Tankyrase 1/2, such as 2X-121. In some embodiments, the additional therapeutic agent includes one or more cyclin-dependent kinase inhibitors, such as CYC-065, CYC-202. In some embodiments, the additional therapeutic agent includes one or more cytosine DNA methyltransferase inhibitors, such as decitabine. In some embodiments, the additional therapeutic agent includes one or more DHFR inhibitors, such as methotrexate. In some embodiments, the additional therapeutic agent includes one or more small ubiquitin related modifier inhibitors, such as TAK-981. In some embodiments, the additional therapeutic agent includes one or more integrin agonists, such as 7HP-349. In some embodiments, the additional therapeutic agent includes one or more sBET inhibitor, such as apabetalone. In some embodiments, the additional therapeutic agent includes one or more BRD4 inhibitors, such as CPI-0610, ABBV-744. In some embodiments, the additional therapeutic agent includes one or more ER1 inhibitors, such as toremifene.

In some embodiments, the additional therapeutic agent includes one or more KRAS inhibitors. In some embodiments, the additional therapeutic agent includes AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), RT11, MRTX-849 (G12C) and K-Ras(G12D)-selective inhibitory peptides, including KRpep-2 (Ac-RRCPLYISYDPVCRR-NH2), KRpep-2d (Ac-RRRRCPLYISYDPVCRRRR-NH2), or any combination thereof.

In some embodiments, the additional therapeutic agent includes one or more inflammation inhibitors, such as pirfenidone. In some embodiments, the additional therapeutic agent includes LYT-100.

In some embodiments, the additional therapeutic agent includes dociparstat sodium. In some embodiments, the additional therapeutic agent includes one or more agents used in the treatment of septic shock, such as nangibotide. In some embodiments, the additional therapeutic agent includes one or more CCR1 antagonists, such as MLN-3897. In some embodiments, the additional therapeutic agent includes one or more agents that target IKKß and NFκB, such as OP-101. In some embodiments, the additional therapeutic agent includes one or more glucocorticoid receptor agonists, such as hydrocortisone or dexamethasone. In some embodiments, the additional therapeutic agent includes one or more immunosuppressants, such as tacrolimus, BXT-10, ibudilast, FP-025, apremilast, abatacept, crizanlizumab, itolizumab, bardoxolone methyl, M-5049. In some embodiments, the additional therapeutic agent includes one or more RIP-1 kinase inhibitors, such as DNL-758. In some embodiments, the additional therapeutic agent includes one or more IL-8 receptor antagonists, such as BMS-986253 (HuMax-IL8). In some embodiments, the additional therapeutic agent includes one or more CD14 inhibitors, such as IC-14. In some embodiments, the additional therapeutic agent includes one or more Dihydroorotate dehydrogenase (DHODH) inhibitors, such as brequinar, PCT-299. In some embodiments, the additional therapeutic agent includes one or more anti-fibrotic agents, such as RT-1840, nintedanib, GB-0139, nintedanib, pamrevlumab. In some embodiments, the additional therapeutic agent includes one or more hepatocyte growth factor (HGF) mimetics, such as SNV-003 (ANG-3777).

In some embodiments, the additional therapeutic agent includes one or more vaccines. In some embodiments, the additional therapeutic agent includes a DNA vaccine, RNA vaccine, live-attenuated vaccine, therapeutic vaccine, prophylactic vaccine, protein based vaccine, or any combination thereof. In some embodiments, the additional therapeutic agent includes a DNA vaccine. In some embodiments, the additional therapeutic agent includes a RNA vaccine. In some embodiments, the additional therapeutic agent includes a live-attenuated vaccine. In some embodiments, the additional therapeutic agent includes a therapeutic vaccine. In some embodiments, the additional therapeutic agent includes a prophylactic vaccine. In some embodiments, the additional therapeutic agent includes a protein based vaccine.

In some embodiments, the additional therapeutic agent includes one or more poxvirus vaccines. In some embodiments, the additional therapeutic agent includes one or more vaccinia virus vaccines. In some embodiments, the additional therapeutic agent includes one or more vaccines effective against variola virus (smallpox), monkeypox virus, or both. In some embodiments, the additional therapeutic agent includes one or more vaccines effective against variola virus (smallpox). In some embodiments, the additional therapeutic agent includes JYNNEOS (also known as Imvamune or Imvanex), ACAM2000, Aventis Pasteur Smallpox Vaccine (APSV), or combinations thereof. In some embodiments, the additional therapeutic agent includes one or more vaccines effective against monkeypox virus. In some embodiments, the additional therapeutic agent includes ACAM2000, MVA-BN, or both. In some embodiments, the additional therapeutic agent includes JYNNEOS. In some embodiments, the additional therapeutic agent includes ACAM2000. In some embodiments, the additional therapeutic agent includes MVA-BN.

In some embodiments, the additional therapeutic agent includes one or more antibodies, for example one or more monoclonal antibodies. In some embodiments, the additional therapeutic agent includes one or more antibodies that binds to a poxvirus. In some embodiments, the additional therapeutic agent includes an anti-CD147 antibody. In some embodiments, the additional therapeutic agent includes meplazumab.

In some embodiments, the additional therapeutic agent includes one or more immunomodulators. Examples of immune-based therapies include toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 modulators; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; polymer polyethyleneimine (PEI); gepon; IL-12; WF-10; VGV-1; MOR-22; BMS-936559; CYT-107, interleukin-15/Fc fusion protein, AM-0015, ALT-803, NIZ-985, NKTR-255, NKTR-262, NKTR-214, normferon, peginterferon alfa-2a, peginterferon alfa-2b, recombinant interleukin-15, Xmab-24306, RPI-MN, STING modulators, RIG-I modulators, NOD2 modulators, SB-9200, and IR-103. In some embodiments, the additional therapeutic agent includes fingolimod, leflunomide, or any combination thereof. In some embodiments, the additional therapeutic agent includes thalidomide. In some embodiments, the additional therapeutic agent includes CD24Fc. In some embodiments, the additional therapeutic agent includes one or more type I IL-1 receptor antagonists, such as anakinra. In some embodiments, the additional therapeutic agent includes one or more TLR4 antagonists, such as EB-05.

In some embodiments, the additional therapeutic agent includes one or more agents for treating a poxvirus infection. In some embodiments, the poxvirus is smallpox virus, vaccinia virus, cowpox virus, rabbitpox virus, orf virus, pseudocowpox virus, bovine paular stomatitis virus, tanapox virus, yaba monkey tumor virus, molluscum contagiosum virus, or monkeypox virus. In some embodiments, the poxvirus is smallpox virus. In some embodiments, the additional therapeutic agent includes cidofovir, brincidofovir (tembexa), tecovirimat or ST-246 (TPOXX), intravenous vaccinia immune globulin (VIGIV), or any combination thereof. In some embodiments, the additional therapeutic agent is tecovirimat, brincidofovir, or cidofovir. In some embodiments, the additional therapeutic agent includes brincidofovir, tecovirimat, or both. In some embodiments, the additional therapeutic agent is cidofovir. In some embodiments, the additional therapeutic agent is brincidofovir. In some embodiments, the additional therapeutic agent is tecovirimat. In some embodiments, the additional therapeutic agent is intravenous vaccinia immune globulin.

In some embodiments, the additional therapeutic agent includes one or more agents for treating a molluscum contagiosum virus infection. In some embodiments, the additional therapeutic agent is cimetidine. In some embodiments, the additional therapeutic agent includes podophyllotoxin, iodine and salicylic acid, potassium hydroxide, tretinoin, cantharidin, imiquimod, or combinations thereof.

In some embodiments, the additional therapeutic agent includes a compound selected from adefovir, tenofovir, adefovir dipivoxil, tenofovir disoproxil, tenofovir disoproxil hemifumarate, tenofovir disoproxil fumarate, tenofovir alafenamide, tenofovir alafenamide hemifumarate, and tenofovir alafenamide fumarate. In some embodiments, the additional therapeutic agent is adefovir. In some embodiments, the additional therapeutic agent is tenofovir. In some embodiments, the additional therapeutic agent is adefovir dipivoxil. In some embodiments, the additional therapeutic agent is tenofovir disoproxil. In some embodiments, the additional therapeutic agent is tenofovir disoproxil hemifumarate. In some embodiments, the additional therapeutic agent is tenofovir disoproxil fumarate. In some embodiments, the additional therapeutic agent is tenofovir alafenamide. In some embodiments, the additional therapeutic agent is tenofovir alafenamide hemifumarate. In some embodiments, the additional therapeutic agent is tenofovir alafenamide fumarate.

The compounds and pharmaceutical compositions described herein can also be used in combination with general care provided to patients with a poxvirus infection, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication (such as acetaminophen), topical steroids or anesthetics (such as lidocaine), antiemetic (such as metoclopramide) and/or antidiarrheal agents, stool softeners, oral antiseptics (such as chlorhexidine mouthwash), analgesic mouthwash (such as those including antihistamine and/or anesthetic), oral antihistamines, topical agents to prevent itching (such as calamine lotion, petroleum jelly, or colloidal oatmeal), vitamin and mineral supplements (including Vitamin K and zinc sulfate), anti-inflammatory agents (such as ibuprofen or steroids), corticosteroids such as methylprednisolone, pain medications (such as gabapentin or opioids), and medications for other common diseases in the patient population, such malaria (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis.

Co-administration of a compound or pharmaceutical composition described herein with one or more other additional therapeutic agents generally refers to simultaneous or sequential administration of the compound or pharmaceutical composition and one or more other additional therapeutic agents described herein, such that therapeutically effective amounts of the compound or pharmaceutical composition and one or more other active therapeutic agents are both present in the body of the patient.

In some embodiments, the compound or pharmaceutically acceptable salt or deuterated analog thereof and the additional therapeutic agents are administered simultaneously. In some embodiments, the pharmaceutical composition and additional therapeutic agent are administered simultaneously. In some embodiments, the compound or pharmaceutically acceptable salt or deuterated analog thereof and the additional therapeutic agent are administered sequentially. In some embodiments, the pharmaceutical composition and the additional therapeutic agent are administered sequentially.

Co-administration includes administration of unit dosages of the compounds or pharmaceutical compositions described herein before or after administration of unit dosages of one or more additional therapeutic agents, for example, administration of the compounds or pharmaceutical compositions described herein within seconds, minutes, or hours of the administration of one or more additional therapeutic agents. For example, a unit dose of a compound or pharmaceutical composition described herein can be administered first, followed within seconds or minutes by administration of a unit dose of one or more additional therapeutic agents. Alternatively, a unit dose of one or more additional therapeutic agents can be administered first, followed by administration of a unit dose of a compound or pharmaceutical composition described herein within seconds or minutes. In some cases, it may be desirable to administer a unit dose of a compound or pharmaceutical composition described herein first, followed, after a period of hours (e.g., 1-12 hours), by administration of a unit dose of one or more other additional therapeutic agents. In other cases, it may be desirable to administer a unit dose of one or more additional therapeutic agents first, followed, after a period of hours (e.g., 1-12 hours), by administration of a unit dose of a compound or pharmaceutical composition described herein.

The combination therapies described herein may provide “synergy” or a “synergistic effect,” i.e. the effect achieved when active ingredients are used together is greater than the sum of the effects that results from using those agents separately. A synergistic effect may be attained when the compounds described herein and the one or more additional therapeutic agents are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds or pharmaceutical compositions described herein are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-viral effect denotes an antiviral effect which is greater than the predicted purely additive effects of the individual compounds of the combination.

Also provided is a compound of Formula I, or a pharmaceutically acceptable salt or deuterated analog thereof, for use in treating a poxvirus infection.

Also provided is a compound of Formula I, or a pharmaceutically acceptable salt or deuterated analog thereof, for use in preventing a poxvirus infection.

In some embodiments, the compound for use is a compound of Formula Ia. In some embodiments, the compound for use is a compound of Formula Ib.

Also provided is a use of a compound of Formula I, or a pharmaceutically acceptable salt or deuterated analog thereof, in the preparation of a medicament for treating a poxvirus infection.

Also provided is a use of a compound of Formula I, or a pharmaceutically acceptable salt or deuterated analog thereof, in the preparation of a medicament for preventing a poxvirus infection.

In some embodiments, the compound for use is a compound of Formula Ia. In some embodiments, the compound for use is a compound of Formula Ib.

Also provided is a kit that includes a compound of Formula I, or a pharmaceutically acceptable salt or deuterated analog thereof, and directions for their use in treating a poxvirus infection.

Also provided is a kit that includes a compound of Formula I, or a pharmaceutically acceptable salt or deuterated analog thereof, and directions for their use in preventing a poxvirus infection.

In some embodiments, the kit includes a compound of Formula Ia. In some embodiments, the kit includes a compound of Formula Ib.

In some embodiments, the kit may contain a single dosage unit and in others multiple dosage units are present, such as the number of dosage units required for a specified regimen or period.

Any formula or structure given herein, including compounds of Formula I, Formula Ia, and Formula Ib, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²p, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes compounds (e.g., compounds of Formula I, Formula Ia, and Formula Ib) in which from 1 to x hydrogens attached to a carbon atom is/are replaced by deuterium, in which x is the number of hydrogens in the molecule. In some embodiments, a deuterated analog of a compound disclosed herein is a compound having one or more hydrogens attached to a carbon of the compound replaced by deuterium. In some embodiments, a deuterated analog is a compound of Formula I having one or more hydrogens attached to a carbon of the compound of Formula I replaced by deuterium. In some embodiments, a deuterated analog is a compound of Formula Ia having one or more hydrogens attached to a carbon of the compound of Formula Ia replaced by deuterium. In some embodiments, a deuterated analog is a compound of Formula Ib having one or more hydrogens attached to a carbon of the compound of Formula Ib replaced by deuterium. In some embodiments, a deuterated analog of a compound disclosed herein is a compound having one hydrogen attached to a carbon of the compound replaced by deuterium.

Such compounds exhibit increased resistance to metabolism and are thus useful for increasing the half-life of any compound described herein when administered to a mammal, particularly a human. See, for example, Foster, “Deuterium Isotope Effects in Studies of Drug Metabolism”, Trends Pharmacol. Sci. 5(12):524-527 (1984). In view of the present disclosure, such compounds are synthesized by means known in the art, for example by employing starting materials in which one or more hydrogens have been replaced by deuterium.

Deuterium labeled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I, Formula Ia and Formula Ib.

### EXAMPLES

### Example 1. In Vitro Antiviral Activity Against Vaccinia Virus

The compound of Formula Ia was tested for in vitro antiviral activity against vaccinia virus (modified vaccinia virus Ankara; MVA) in BHK-21 baby hamster kidney cells. The compound of Formula Ia was serially diluted using eight half-log dilutions in test media (MEM+5% fetal bovine serum and 50 μg/mL). Each dilution was added to 5 wells of a 96-well plate with 80-100% confluent cells. Three wells of each dilution were infected with virus, and two wells remained uninfected as toxicity controls. Six wells were infected and untreated as virus controls, and six wells were uninfected and untreated as cell controls. Virus was prepared to achieve a MOI of 0.02. Cidofovir was tested in parallel as a positive control. Plates were incubated at 37±2° C., 5% CO₂ for 3 days. On day 3 post-infection, untreated virus control wells reached maximum CPE and plates were stained with neutral red dye for approximately 2 hours (+15 minutes). Supernatant dye was removed and wells rinsed with phosphate-buffered saline (PBS). The incorporated dye was extracted in 50:50 Sorensen citrate buffer/ethanol for >30 minutes and the optical density was read on a spectrophotometer at 540 nm. Optical densities were converted to percent of cell controls and normalized to the virus control. The concentration of the compound of Formula Ia required to inhibit CPE by 50% (EC₅₀) was calculated by regression analysis. The concentration of the compound of Formula Ia causing 50% cell death in the absence of virus was similarly calculated (CC₅₀). The selective index (SI) is the CC₅₀ divided by EC₅₀.

The in vitro antiviral results of the compound of Formula Ia against vaccinia virus are shown in Table 1. The in vitro antiviral results of the compound of Formula Ia against vaccinia virus for Tests 1-3 are shown in Table 1A.

TABLE 1

| | Compound of Formula Ia | | | Cidofovir control | | |
|---|---|---|---|---|---|---|
| | EC₅₀ | CC₅₀ | | EC₅₀ | CC₅₀ | |
| | (μM) | (μM) | SI | (μM) | (μM) | SI |
| Test 1 | 2.3 | 23 | 10 | 2.6 | >100 | >38.5 |
| Test 2 | 0.55 | 2.5 | 4.5 | 7.2 | >100 | >14 |
| Average | 1.43 | 12.8 | 7.25 | 4.9 | >100 | >14 |
| Standard | 0.88 | 10.3 | 2.75 | 2.3 | — | — |
| deviation | | | | | | |

TABLE 1A

| | Compound of Formula Ia | | | Cidofovir control | | |
|---|---|---|---|---|---|---|
| | EC₅₀ | CC₅₀ | | EC₅₀ | CC₅₀ | |
| | (μM) | (μM) | SI | (μM) | (μM) | SI |
| Test 1 | 2.3 | 23 | 10 | 2.6 | >100 | >38.5 |
| Test 2 | 0.55 | 2.5 | 4.5 | 7.2 | >100 | >13.9 |
| Test 3 | 3.3 | >50 | >15 | 6.3 | 73 | 12 |
| Average | 2.80 | >2.5 | >4.5 | 5.37 | >73 | >12 |
| Standard | 0.50 | — | — | 2.43 | — | — |
| deviation | | | | | | |

### Example 2. Vaccinia Virus Cellomics Assay

The compound of Formula Ia was tested for ability to inhibit vaccinia virus replication by monitoring the levels of vaccinia virus fusion protein A27L using a high-throughput image-based Cellomics assay.

BHK-21 cells (baby hamster kidney cells strain 21, ATCC, CCL-10) were maintained in EMEM medium (ATCC, 30-2003) supplemented with 10% FBS (Corning, 35-011-CV) and 1% Penicillin-Streptomycin (Corning, 30-002-CI). Prior to confluence, BHK21 cells were dislodged with 0.25% Trypsin (Gibco, 25200-056), neutralized by culture media, and resuspended in assay medium (EMEM, 2% FBS, 1% Penicillin-Streptomycin). Cell density was adjusted to 450,000 cells/mL, and 20 μl/well of the cell suspension were added into 384-well microplates (cellomics plates: Greiner, 781946; cytotoxicity plates: Corning, 3765) and incubated overnight at 37° C. 5% CO₂.

Ten-dose, 3-fold serial dilutions of the compound of Formula Ia were prepared at starting concentrations of 10 mM in 100% DMSO. 150 nL of compounds were spotted in quadruplicates onto the plates using an HP D300e dispenser. The final starting concentration in the assay was 50 μM DMSO (no compound, negative control) and brincidofovir and/or tecovirimat (positive controls) were included on each microplate. Vaccinia virus (MVA strain: ATCC, VR1508) was diluted in assay media and added to the Cellomics plates at 10 μL/well to reach an MOI of 0.04. For cytotoxicity plates, 10 μL/well of assay media (no virus) was added to all wells. Plates were then incubated at 37° C. 5% CO₂ for 2 days.

Cell culture medium was aspirated and cells were fixed with 40 μL/well of a paraformaldehyde solution (Electron Microscopy Sciences, 15712-S) diluted to 4% in DPBS (Corning, 21-031-CM). After a 30-minute incubation at room temperature, plates were washed with 80 μL/well of DPBS once. 7.5 μg/mL of anti-vaccinia antibody (Abcam, 35219) was prepared in staining buffer (PBS+0.1% Triton X-100+0.2% Gelatin) and 25 μL/well were added into each well. Plates were incubated at room temperature for an hour. After washing the plates with 80 μL/well PBS, 25 μL/well of a 1:1000 solution of secondary antibody (Alexa Fluor 488 goat anti-rabbit: Jackson Immuno Research, 711-095-152) and DAPI (Thermo Fisher Scientific, 62248) in staining buffer were added into wells, and plates were incubated in the dark at room temperature for 1 hour. Plates were washed once with 80 μL/well of PBS prior to sealing with a black adhesive seal. Fluorescence was measured on a Cellomics plate reader. For the cytotoxicity plates, 30 μL/well of CellTiter-Glo (Promega, G7570) was added to the plates, followed by a 15-min incubation at RT before quantification of luminescence on an Envision plate reader.

Data analysis was carried out using the Thermo Scientific HCS Studio software. Cells were identified using the DAPI nuclear stain and thresholds were set to filter cells out based on shape and size. A second threshold based on green fluorescence intensity (detection of vaccinia virus fusion protein A27L) was set to identify vaccinia infected cells. Data was reported as the average fluorescence area of vaccinia infected cells per object. EC₅₀ values were defined as the compound concentration that caused a 50% decrease in the average fluorescence area and was calculated using a sigmoidal dose-response model to generate curve fits. CC₅₀ values for cytotoxicity were calculated similar way using the luminescent signals normalized by DMSO controls.

The assay results for the compound of Formula Ia (n=3) are shown in Table 2.

TABLE 2

| | EC₅₀ | |
|---|---|---|
| | (μM) | CC₅₀ (μM) |
| Compound of | 0.726 | 26.1 |
| Formula Ia | | |

The assay results for the compound of Formula Ia (n=4) are shown in Table 2A.

TABLE 2A

| | Average | |
|---|---|---|
| | EC₅₀ ± | |
| | SD (μM) | CC₅₀ ± SD (μM) |
| Compound of | 0.773 ± 0.131 | 28.8 ± 11.5 |
| Formula Ia | | |

SD = standard deviation

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter and is not intended to limit the appended claims to the specific embodiments illustrated.

1. A method of treating a poxvirus infection in a patient in need thereof, wherein the method comprises administering to the patient a therapeutically effective amount of a compound of Formula I, Formula Ia, or Formula Ib: 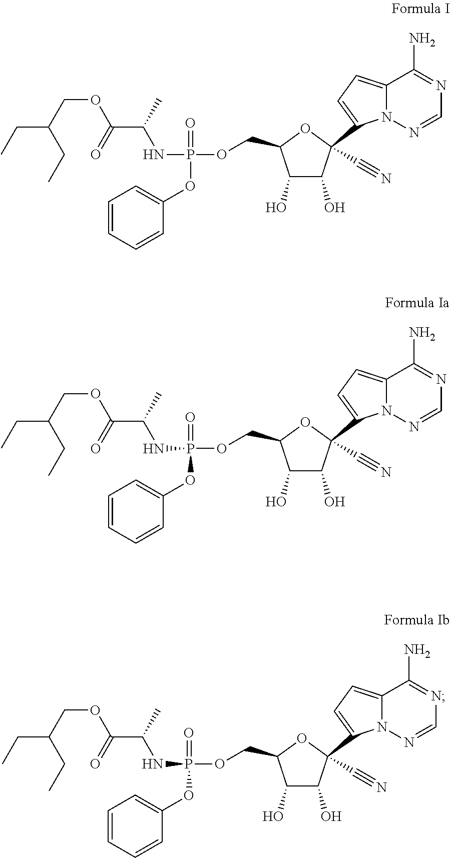 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1 ; CCC(CC)COC(=O)[C@H](C)N[P@](=O)(OC[C@H]1O[C@@](C#N)(c2ccc3c(N)ncnn23)[C@H](O)[C@@H]1O)Oc1ccccc1 ; CCC(CC)COC(=O)[C@H](C)N[P@@](=O)(OC[C@H]1O[C@@](C#N)(c2ccc3c(N)ncnn23)[C@H](O)[C@@H]1O)Oc1ccccc1]
or a pharmaceutically acceptable salt or deuterated analog thereof.

2. (canceled)

3. The method of claim 1, wherein the compound of Formula I, Formula Ia, or Formula Ib is administered.

4-8. (canceled)

9. The method of claim 1, wherein the compound or the pharmaceutically acceptable salt or deuterated analog thereof is administered orally.

10. The method of claim 1, wherein the compound or the pharmaceutically acceptable salt or deuterated analog thereof is administered parenterally.

11. The method of claim 1, wherein the compound or the pharmaceutically acceptable salt or deuterated analog thereof is administered once daily.

12. (canceled)

13. (canceled)

14. The method of claim 1, wherein the compound or the pharmaceutically acceptable salt or deuterated analog thereof is administered at a dosage of 5 mg to 300 mg.

15. (canceled)

16. The method of claim 1, wherein the compound or the pharmaceutically acceptable salt or deuterated analog thereof is administered at a dosage of 0.1 mg/kg to 15 mg/kg.

17. The method of claim 1, further comprising administering to the patient a therapeutically effective amount of an additional therapeutic agent.

18-20. (canceled)

21. A method of treating a poxvirus infection in a patient in need thereof, wherein the method comprises administering to the patient a pharmaceutical composition comprising a compound of Formula I, Formula Ia, or Formula Ib: 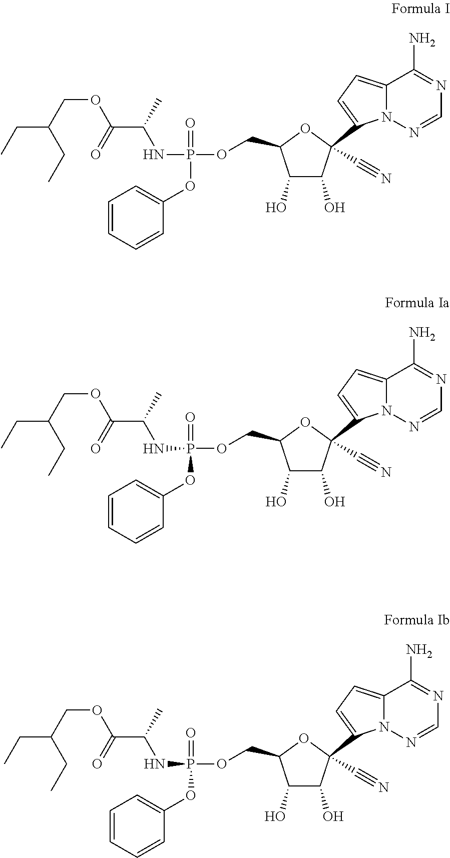 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1 ; CCC(CC)COC(=O)[C@H](C)N[P@](=O)(OC[C@H]1O[C@@](C#N)(c2ccc3c(N)ncnn23)[C@H](O)[C@@H]1O)Oc1ccccc1 ; CCC(CC)COC(=O)[C@H](C)N[P@@](=O)(OC[C@H]1O[C@@](C#N)(c2ccc3c(N)ncnn23)[C@H](O)[C@@H]1O)Oc1ccccc1]
or a pharmaceutically acceptable salt or deuterated analog thereof, and one or more pharmaceutically acceptable carriers.

22-34. (canceled)

35. The method of claim 1, wherein the poxvirus infection is an orthopox virus infection.

36. The method of claim 1, wherein the poxvirus infection is a camelpox virus infection, cowpox virus infection, ectromelia virus infection, horsepox virus infection, monkeypox virus infection, raccoonpox virus infection, skunkpox virus infection, taterapox virus infection, uasin gishu virus infection, vaccinia virus infection, variola virus infection, or volepox virus infection.

37-40. (canceled)

41. The method of claim 1, wherein the poxvirus infection is a parapoxvirus infection.

42. (canceled)

43. (canceled)

44. The method of claim 1, wherein the poxvirus infection is a molluscipoxvirus infection.

45. (canceled)

46. The method of claim 1, wherein the poxvirus infection is a yatapoxvirus infection.

47. (canceled)

48. The method of claim 1, wherein poxvirus infection is a capripoxvirus infection.

49. (canceled)

50. The method of claim 1, wherein the poxvirus infection is a suipoxvirus infection.

51. (canceled)

52. The method of claim 1, wherein the poxvirus infection is a leporipoxvirus infection.

53. (canceled)

54. The method of claim 1, wherein the poxvirus infection is an avipoxvirus infection.

55. (canceled)

56. (canceled)

57. The method of claim 1, wherein the patient is a human.

58-61. (canceled)

62. A kit comprising a compound of Formula I: 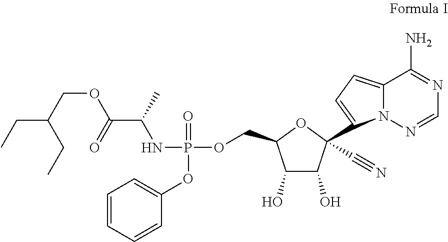 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]
or a pharmaceutically acceptable salt or deuterated analog thereof, and directions for their use in treating a poxvirus infection.

63. (canceled)
